(19) United States
(12) Patent Application Publication
(10) Pub. No.: US 2005/0196773 A1
(43) Pub. Date: Sep. 8, 2005

(54) Predicting upper aerodigestive tract cancer

(76) Inventors: David Sidransky, Baltimore, MD (US); Li Mao, Houston, TX (US); Hening Ren, Houston, TX (US)

Correspondence Address: BANNER & WITCOFF, 1001 G STREET N W, SUITE 1100, WASHINGTON, DC, 20001, US

(21) Appl. No.: 10/986,161

(22) Filed: Nov. 12, 2004

Related U.S. Application Data
(60) Provisional application No. 60/519,340, filed on Nov. 12, 2003.

(51) Int. Cl. C12Q 1/68; G06F 19/00; G01N 33/48; G01N 33/50

(52) U.S. Cl. 435/6; 702/20

(57) ABSTRACT

Cancer screening models based on analysis of mass spectroscopy data can be used to predict upper aerodigestive tract cancer, including lung and head and neck cancers. Models can be generated by comparing spectral weight values obtained from upper aerodigestive tract cancer patients and from patients at high risk for such cancer. Predictor or covariate values identify spectral weight values associated with upper aerodigestive tract cancer.

This application claims the benefit of and incorporates by reference provisional application Ser. No. 60/519,340 filed Nov. 12, 2003.

### FIELD OF THE INVENTION

The present invention generally relates to cancer diagnosis. The invention relates more specifically to methods of early prediction and detection of cancers in a human or animal subject based on mass spectra data.

### BACKGROUND OF THE INVENTION

The approaches described in this section could be pursued, but are not necessarily approaches that have been previously conceived or pursued. Therefore, unless otherwise indicated herein, the approaches described in this section are not prior art to the claims in this application and are not admitted to be prior art by inclusion in this section.

Lung cancer is the leading cause of cancer-related death in the United States and other major industrialized nations. Despite extensive efforts made in development of diagnostic and therapeutic methods during the past three decades, the overall rate of survival, measured at five years after diagnosis, remains low. The low survival rate is due mainly to the lack of effective methods to diagnose lung cancer early enough for cure, and lack of regimens to sufficiently prolong quality of life of patients with advanced stages of lung cancer. In current practice, only 15% of patients with lung cancers are diagnosed when tumors are at a localized stage, and a five-year survival rate of 50% is expected for this population. Once tumors spread out of the local region, the outcome is extremely poor.

Head and neck squamous cell carcinoma (“HNSCC”) is also a major health problem worldwide with over 500,000 cases each year. The overall 5-year survival for patients with the disease is only 50%.

Development of lung and head and neck cancers requires repeated introduction of carcinogens, typically from tobacco smoke, in the upper aero-digestive tract over a long period time. The development process (“carcinogenesis”) can take many years and results in accumulation of multiple molecular abnormalities in cells, which are the basis of malignant transformation and tumor progression.

Evidence has emerged to demonstrate that genetic abnormalities occur in the early carcinogenic process in the lungs and oral cavity of chronic smokers, and certain abnormalities may persist for many years after smoking cessation. A number of genetic and molecular alterations, such as mutations in the p53 tumor suppressor gene and K-ras protooncogene, promoter hypermethylation of the p16 tumor suppressor gene, and loss of heterozygosity in multiple critical chromosome regions, have been frequently identified in the early stages of the diseases.

Accordingly, a number of investigators have been exploring the possibility of using these alterations as biomarkers in early detection and risk assessment of lung and head and neck cancers. With the completion of human genome mapping and advances in high throughput technologies, the discovery of molecular alterations in the carcinogenic process is accelerating. A substantial effort is now underway to conduct large-scale cooperative discoveries and validations of biomarkers for early cancer diagnosis, such as the Early Detection Research Network (EDRN) sponsored by National Cancer Institute in the United States. Molecular marker-based novel diagnostic strategies are expected to be developed and introduced into clinical practice to augment current inefficient tools in diagnosing patients with early stage lung and head and neck cancers.

cDNA microarrays have also been explored for molecular classification of human malignancies and have shown promising results. However, the strategy is hardly practicable in early diagnosis of lung, head and neck cancer because it requires adequate biological materials with sufficient malignant cells.

Protein/peptide pattern recognition in serum recently has been used for high throughput diagnosis of ovarian cancer. This mass spectrometer based test has shown an extremely high detection sensitivity and specificity in predicting patients with and without ovary cancer.

Based on current knowledge, it appears that no single marker can make a sensitive and specific diagnosis of early stage lung cancers. Accordingly, analyzing more than one biomarker may be necessary to achieve a clinically acceptable sensitivity and specificity for early lung cancer diagnosis.

Based on the foregoing, there is a clear need for an improved method of predicting and making early diagnosis of cancer, such as cancers of the lungs, head and neck. It is also desirable to have a method of predicting or making an early diagnosis of cancer from results primarily based on data analysis of compounds in a relatively small tissue sample.

### BRIEF DESCRIPTION OF THE DRAWINGS

The present invention is illustrated by way of example, and not by way of limitation, in the figures of the accompanying drawings.

FIG. 1A is a flow diagram that illustrates an overview of one embodiment of a method for generating a cancer-screening model.

FIG. 1B is a data flow diagram that illustrates use of data and related elements in the method illustrated in FIG. 1A.

FIG. 2A is a flow diagram that illustrates an overview of one embodiment of a method for predicting lung, head and neck cancer in mammals.

FIG. 2B is a data flow diagram that illustrates use of data and related elements in the method illustrated in FIG. 2A.

FIG. 3 shows area under the receiver operating characteristic (ROC) curves for false-positive rates between 0 and 1 (solid line) and area under the ROC curves for false positive rates between 0 and 0.10 (dashed line) plotted against the number of features (P) used in linear discriminant analysis (LDA). Vertical lines show the maximum occurrence for each curve. Data includes all head and neck cancer patients for each value of P. Area under the ROC curves was calculated using the cross-validation procedure described herein.

FIG. 4 shows average ROC curves for observed data (solid line) and the null hypothesis (dashed line). The thick dashed diagonal line represents the expected ROC curve under the null hypothesis in which X and Y are independent and there is no information in the spectra the outcomes. Gray dashed lines represent null permutations, and gray solid lines represent spectral data permutations. Numbers shown on the curves represent the value of LDA tuning parameters that yielded specificity and sensitivity represented by the respective black squares and generated by the cross-validation procedure described herein.

FIG. 5 shows differences in average mass spectra between case patients (solid line) and control subjects (dashed line). Average spectra were derived from 99 head and neck cancer patients and 143 control subjects. The frequency at which features were selected during the 200 random divisions of the data into training and test sets is shown in the bottom panel. The range of y-axis (0% to 100%) is for spectral peaks occurring in case patients but not control subjects.

FIG. 6 illustrates a block diagram of a hardware environment that may be used according to an illustrative embodiment of the invention.

### DETAILED DESCRIPTION OF THE INVENTION

Methods and apparatus for detecting cancers in mammals based on mass spectra data is described. Methods of the present invention can be carried out to detect the presence of cancer in a human or animal subject by analyzing mass spectral data from the serum or blood of the subject for an enhanced or reduced level of one or more molecular species as compared to the mass spectral data of normal subjects.

In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention.

Embodiments are described herein according to the following outline:

| 1.0 | General Overview | |
|---|---|---|
| 2.0 | Method and Apparatus for Predicting Cancer | |
| | 2.1 | Generating Sample Data |
| | 2.2 | Creating Prediction Model |
| | 2.3 | Performing Predictions |
| | 2.4 | Empirical Results |
| | 2.5 | Representing Prediction as a Regression Problem |

| | |
|---|---|
| 3.0 | Implementation Mechanisms - Computer Hardware Overview |
| 4.0 | Extensions and Alternatives |

1.0 General Overview

The needs identified in the foregoing Background, and other needs and objects that will become apparent for the following description, are achieved in the present invention, which comprises, in one aspect, a method for predicting lung, head and neck cancers in mammals. “Predicting,” as used herein, includes diagnosing, prognosing the course of, and prognosing the likelihood of developing such cancers. Lung cancers include small cell carcinomas and non-small cell carcinomas (e.g., squamous cell carcinomas, adenocarcinomas, and large cell carcinomas). “Head and neck cancer,” as is known in the art, includes all malignant tumors which occur on the head and neck, including the mouth, nasal passages, eye, ear, larynx, pharynx, and skull base. Examples of head and neck cancers include, but are not limited to, hypopharyngeal cancer, laryngeal cancer, lip cancer, oral cavity cancer, malignant melanoma, nasopharyngeal cancer, oropharyngeal cancer, paranasal sinus cancer, nasal cavity cancer, salivary gland cancer, and thyroid cancer.

According to one embodiment, spectra sample data are generated from sera obtained from a human population with known pathology with respect to lung, head, or neck cancer. The sample data are divided into a training data set and a test data set. A subset of the sample data values is selected from the training set. Feature extraction is performed on the subset, to further select top spectral weight values. Linear discriminant analysis is then applied to the selected spectral weights of the sample data values, resulting in generating one or more estimated parameter values associated with a conditional distribution. That is, the model generates sample data values associated with the cancer-positive human population from which the sera was obtained. The estimated parameter values are modified by identifying one or more true positives and false positives among them. As a result, a predictive model is created that can be used to classify each sample in the test data, or any other spectra data sample, as representing either a carcinogenic or non-carcinogenic individual.

In one feature of the process, functional discriminant analysis is used for data analysis in a two-stage setting. In particular, a panel of samples is used for training purposes to identify potential profiles that distinguish individuals with cancer from healthy individuals. A second panel derived from different individuals is used for testing purposes to validate the findings generated from the training set. Unlike gene expression data analysis, in which individual genes serve as index values, in mass spectrometer data analysis, each spectra value is continuous. Therefore, the functional form of linear discriminant analysis is used, coupled with feature selection to identify molecules with specific spectra values for optimal class prediction. Accurate prediction is defined as correctly identifying the percentage of individuals with cancer and healthy individuals. After validation of the model against the test data, the model may be used to predict cancer in other populations by matching the model to new data sets.

Using, for example, matrix assisted laser desorption/ionization (“MALDI”) or matrix-assisted laser desorption/ionization-time-of flight mass spectrometry (MALDI-TOFMS), distinct protein/peptide or other molecular patterns may be identified in serum that indicate individuals with lung or head and neck cancers and healthy individuals. In combination with powerful computer-based analytic tools, hundreds of samples may be handled and diagnostic information may be obtained in a relatively brief time. It is understood that the invention also encompasses other forms of profiling, including surface enhanced laser desorption/ionization (SELDI), and any other form of MALDI. In another aspect, the invention encompasses a specific molecule or molecules whose increased or decreased level in blood or serum in individuals with or at risk of cancer, as compared to normal individuals, is indicative of or predictive of cancer. In other aspects, the invention encompasses a computer apparatus, a computer readable medium, and a carrier wave configured to carry out the foregoing steps.

Determination of cancer prediction models of the invention is described by example below. Such cancer prediction models comprise a pattern of cancer predictor spectral weight values which correspond to identifying spectral weights. Identifying spectral weights include 5, 10, 12, 15, 20, 45, 47, 54, 64, and 111 kd. Prediction models for upper aerodigestive tract cancers preferably include a cancer predictor spectral weight value corresponding to 111 kd, however, prediction models of the invention can include cancer predictor spectral weight values corresponding to any combination of 2, 3, 4, 5, 6, 7, 8, or 9 of these identifying spectral weights or to all ten. Those of skill in the art will understand that the precise identifying spectral weights in a model (or in a test sample) may deviate slightly from 5, 10, 12, 15, 20, 45, 47, 54, 64, or 111 kd because of inherent experimental error in the particular instrument used to determine the weights.

Sample data for use in generating cancer prediction models of the invention, or for use in predicting upper aerodigestive tract cancer, can be obtained from biological samples such as serum, sputum, bronchial lavage samples, or biopsy samples. Control populations for use in generating cancer prediction models preferably include individuals at high risk for developing an upper aerodigestive tract cancer (e.g., heavy smokers) but who have been clinically determined not to have an aerodigestive tract cancer. The presence or absence of upper aerodigestive tract cancers typically is based on a clinical history and a physical examination, which may include diagnostic tests such as X-rays, CT or MRI scans, blood tests, bronchial lavage, and biopsies. Preferably each individual in the control population is at high risk for, but does not have, an upper aerodigestive tract cancer.

2.0 Method and Apparatus for Predicting Cancer

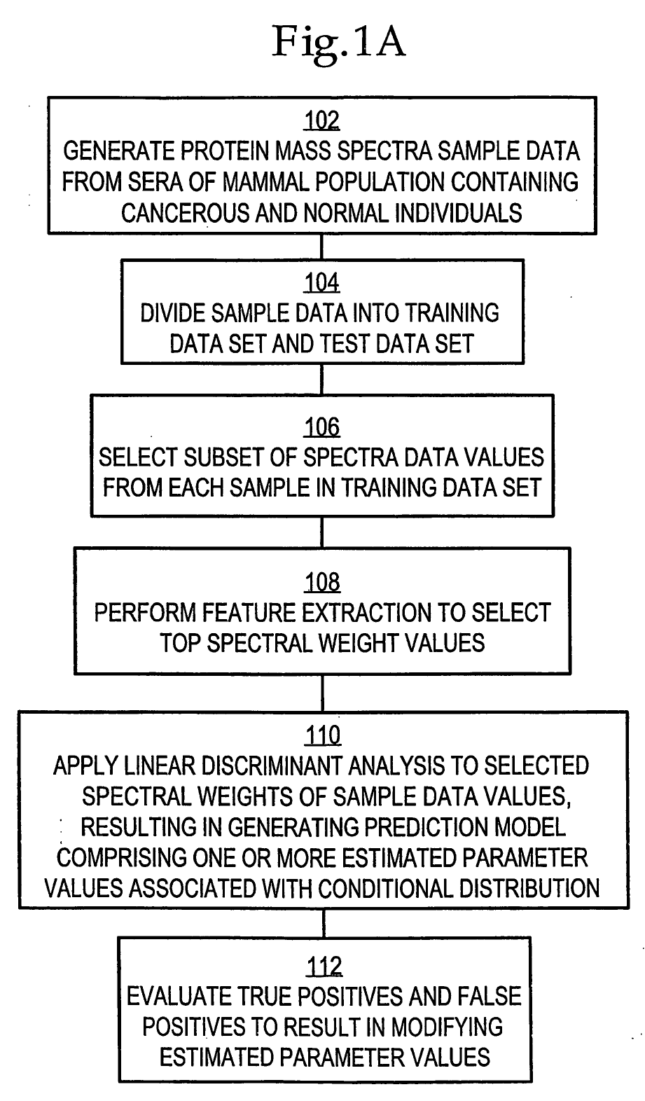

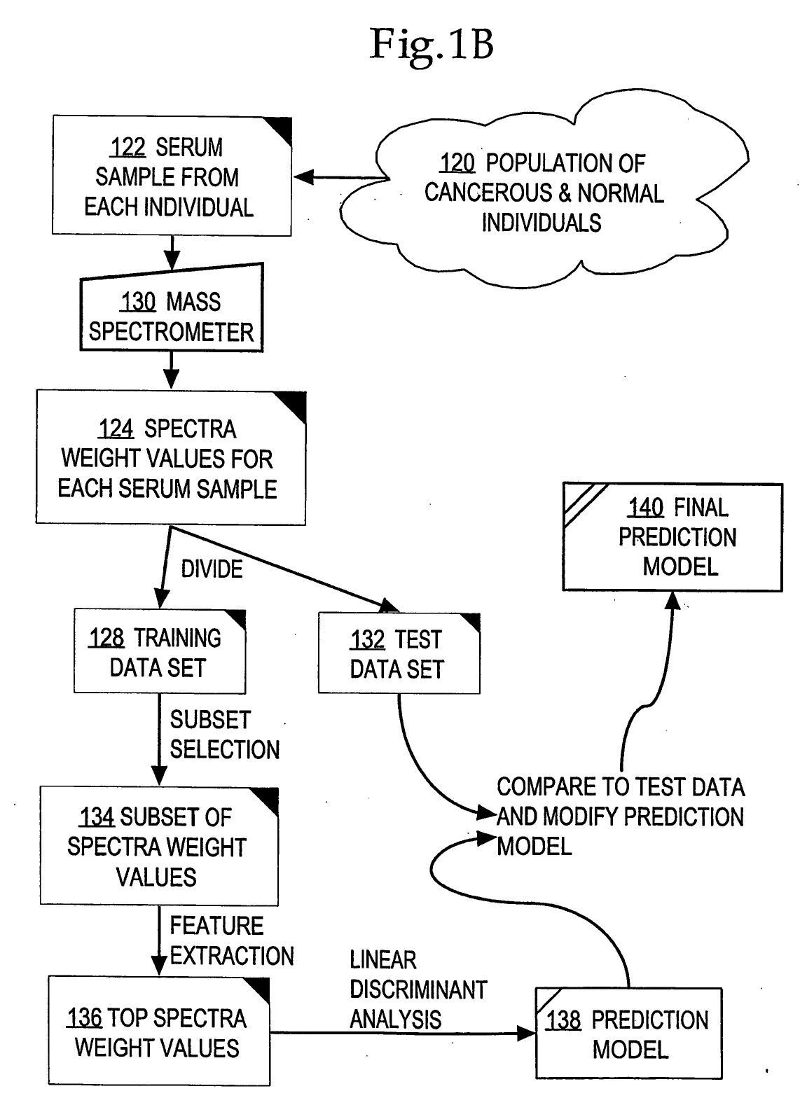

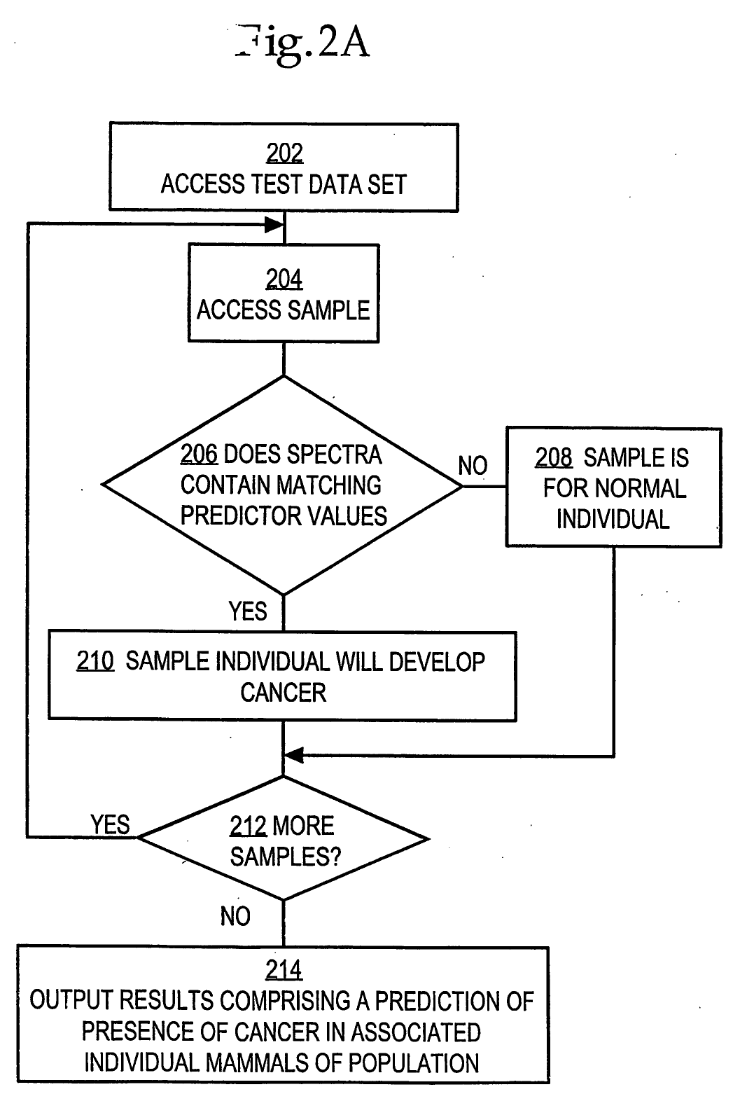

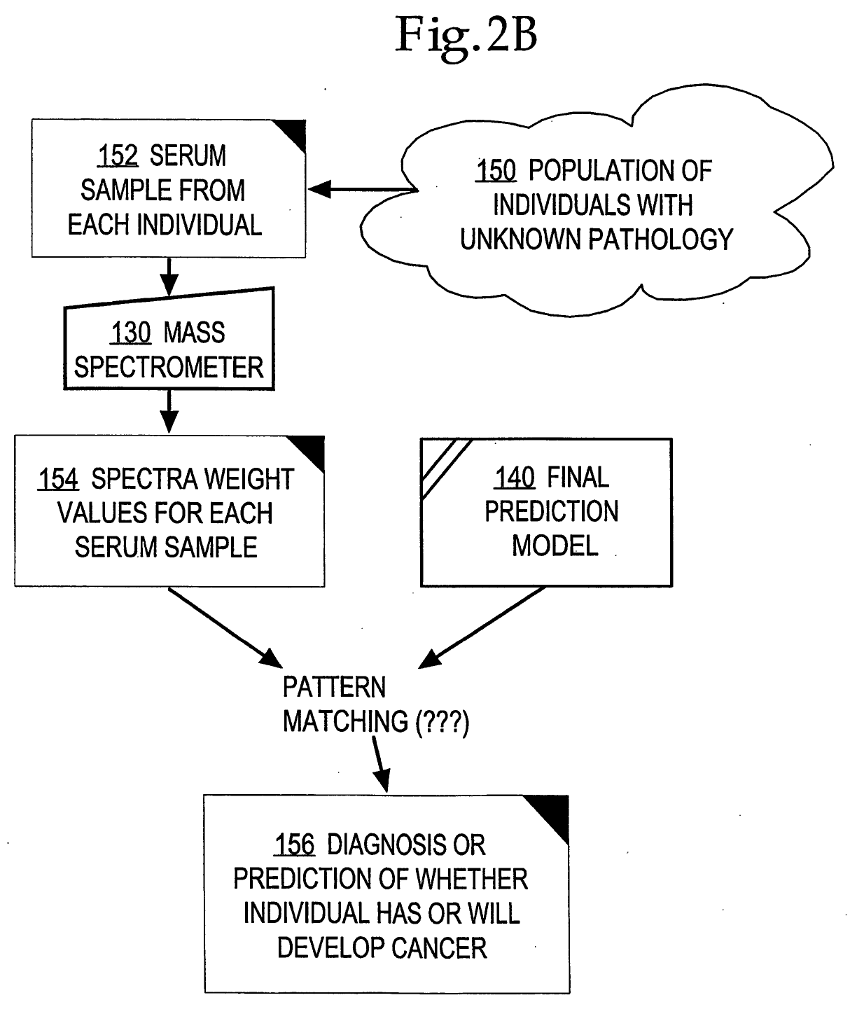

Example embodiments are now described with respect to FIG. 1A, FIG. 1B, FIG. 2A, and FIG. 2B. FIG. 1A is flow diagram that illustrates an overview of an illustrative embodiment of a method for generating a cancer-screening model. FIG. 1B is a data flow diagram that illustrates use of data and related elements in the method of FIG. 1A. FIG. 2A is a flow diagram that illustrates an overview of an illustrative embodiment of a method for predicting lung, head and neck cancer in mammals. FIG. 2B is a data flow diagram that illustrates use of data and related elements in the method of FIG. 2A.

2.1 Generating Sample Data

Referring first to FIG. 1A, in block 102, spectra sample data is generated from sera of a sample population. As shown in FIG. 1B, a population 120 of individuals who are both cancerous and normal yields a serum sample 122 from each individual. The serum sample 122 is applied to a mass spectrometer 130 to result in generating spectral weight values for each serum sample 124.

For example, MALDI-TOFMS is used to generate a spectra sample data set representing distinct protein/peptide patterns in serum. In one clinical investigation, sera from patients with lung or head and neck cancers or healthy controls were obtained before surgical procedures. All final diagnoses were confirmed by histopathology and all controls were heavy smokers but without evidence of lung or head and neck cancer based on clinical presentation and CT scan examination.

The sera were prepared for evaluation by the mass spectrometer by making a matrix of serum samples. The mass spectrometer matrix contained 50% saturated sinapinic acid in 30% acetonitrile-1% trifluoroacetic acid. The serum was diluted 1:1000 in 0.1% n-Octyl β3-D-Glucopyranoside. Five μl of the matrix was placed on each defined area of a sample plate with 384 defined areas and 0.5 μl serum from each individual was added to the defined areas followed by air dry. Samples and their locations on the sample plates were recorded for accurate data interpretation. An Axima-CFR MALDI-TOF mass spectrometer manufactured by Kratos Analytical Inc. was used. The instrument was set as following: tuner mode, linear; mass range, 0 to 180,000; laser power, 90; profile, 300; shots per spot, 5. The output of the mass spectrometer was stored in computer storage in the form of a sample data set.

2.2 Creating Prediction Model

A use of the process described herein is to classify the spectra data values into one of a plurality of binary outcomes that represent normal individuals and individuals that will develop squamous cell carcinoma (“SCC”) of the lung, head or neck. For purposes of mathematical analysis, the spectra data values are denoted X and the outcomes are denoted Y. The process herein seeks to use the spectra data values to predict these outcomes. Each spectra X typically comprises a large plurality of values, denoted P. For example, in one investigation, spectra were digitized at P=284,027 spectra data values in each individual spectrum.

The data can be simplified by optionally considering only every 100th value in the individual spectra. This considerably reduces the complexity and computing time without affecting the final results.

The process herein assumes that the outcome values, the spectra values, and their distribution derive from random processes. The randomness is believed to arise from sampling techniques, measurement errors, and because the naturally occurring compounds under study are inherently random. Based on this assumption, the spectra values may be viewed as predictors or covariates. The individual spectra values (or “spectral weight values”) are denoted as X₁, . . . ,Xₚ.

Spectral values can be log transformed to lessen the mean-variance dependence. To predict outcomes using mass spectra, log transformed spectra can be designated as predictors or covariates denoted, for example, as X═X₁, . . . X₂₈₄₀.

The process herein is directed not to fitting a model and interpreting parameters, but to predicting outcomes. Thus, the process herein seeks to partition the covariates into those for which normal morphology is predicted, and those for which SCC is predicted. The latter covariates are termed “predictors” or “classifiers.”

In one approach, the classifiers could be identified or trained based on data for which both outcome and covariates are known. However, in another approach, the number of covariates is much larger than the number of outcomes, and therefore a classifier that predicts perfectly for the training data may be constructed.

Cross-validation may be used to assess how well the classifier performs. Accordingly, in block 104, the sample data set is divided into a training data set and test data set. As seen in FIG. 1B, the spectral weight values for each serum sample 124 are divided into training data set 128 and test data set 132. In one investigation, two-thirds of the data was randomly selected as a training data set, and the other one-third comprised the test data set, and the procedure herein was repeated 200 times.

In block 106, a subset of sample spectra data values are selected from each sample in the training set. In FIG. 1B, the subset selection operation results in creating a subset of spectral weight values 134. For example, as discussed above, in one investigation in which each individual sample comprised 284,027 spectra data values, only every 100th value in the individual spectra was considered. This approach considerably reduces computing time, and is not believed to affect the accuracy of predictive results.

In block 108, feature extraction is performed to select top spectral weight values from among those that are considered in each sample. In FIG. 1B, feature extraction results in creating top spectral weight values 136. This approach reduces the number of covariates and improves results from subsequent analytical steps. In one investigation, feature extraction involved using the training data to calculate t-statistics, using an equivalent across-group-variance/within-group-variance ratio, and comparing the normal and SCC spectral weight values; the top 45 spectral weight values with the highest t-statistics were then used.

Specifically, with 338 samples and 2840 predictors, a simple feature selection procedure, equivalent to the t-test, was used. The procedure is based on the across-group-variance to within-group-variance ratio, and comparing the normal and cancer values. All spectral values are ranked and the top 45 chosen for linear discriminant analysis (LDA).

In block 110, linear discriminant analysis is applied to the selected spectral weight values of the sample data values. As a result, a prediction model is generated comprising one or more estimated parameter values that are associated with a conditional distribution, as indicated by prediction model 138 of FIG. 1B. That is, the model generates sample data values associated with the cancer-positive human population from which the sera was obtained.

Linear discriminant analysis (LDA) is a classification procedure available in many commercial statistical analysis software applications. For example, the R and S-Plus software packages provide LDA. LDA is described in Ripley B. D. (1996) Pattern Recognition and Neural Networks, Cambridge, U.K. Cambridge University Press. Methods similar to LDA have been used in classification problems using the microarray technology, as described in Golub et al. (1999) “Molecular classification of cancer: Class discovery and class prediction by gene expression monitoring” Science 286, 531-537. Further, LDA has been shown to outperform more elaborate procedures in the context of micro array data in Dudoit, S., Fridlyand, and Speed, T. P. (2002) “Comparison of discrimination methods for the classification of tumors using gene expression data” Journal of the American Statistical Association 97, 77-87.

In one embodiment, use of LDA in block 110 assumes that conditional of Y, the X follow a multivariate normal distribution. Therefore, to predict Y for a particular value of X, the process herein finds a value of Y that maximizes the posterior probability of observing X given that value of Y.

Optionally, in block 112 the estimated parameter values are modified by identifying one or more true positives and false positives among them.

In other applications of LDA, prior probability values are commonly assigned to each of the values of Y. The prior probabilities can be used to control the false positive rates since they affect the posterior probabilities in a direct way. The training data is used to estimate the parameters, mean and covariance matrix, associated with each of the conditional distributions.

2.3 Performing Predictions

A process of performing predictions using the model generated in the process of FIG. 1A is now described, with reference to FIG. 2A.

In block 202, a test data set is accessed, for example, by accessing data values stored in computer storage. In block 204, a first sample value is accessed. The sample value typically comprises a large plurality of individual spectra values.

In block 206, a test is performed to determine whether the first sample value contains any spectral weight values that match the estimated parameter values from the cancer prediction model that was developed in the process of FIG. 1A. If not, then control transfers to block 208, in which the sample is considered as associated with a normal individual. If matching spectral weight values are found, then in block 210 the sample is regarded as representing an individual who will develop cancer. Generally, a matching spectral weight value for a particular spectral peak is within 25% or higher of the cancer prediction model peak, more preferably within 20% or higher, even more preferably, within 15% or higher, yet more preferably, within 10% or higher and, most preferably, within 5% or higher. The above method can apply with respect to at least one peak, two three, four, five, seven, ten, fifteen, twenty, twenty five, thirty or fifty or more peaks assessed in combination. Block 208 and block 210 may involve storing an appropriate data flag in a database in association with a record representing an individual. Those of skill in the art will appreciate that as the matching spectral weight value for a particular spectral peak approaches the spectral weight value for the cancer prediction model peak that the likelihood of a correct result increases. The percentages recited herein are guidelines that have been found to be useful based on successful tests and analysis. However, lower or higher percentages may alternatively be used depending on the margin of error desired. Similarly, applying the method to one peak or to many peaks is also within the scope of the present invention.

Alternatively, to determine whether an individual will develop cancer, the mass spectral data of the sample in block 206 may be compared to the non-cancer (or normal) prediction model. If non-matching spectral values are found, then in block 210 the sample is regarded as representing an individual who will develop cancer. Generally, a non-matching spectral value for a particular spectral peak is 50% or higher than the peak of the non-cancer prediction model peak, more preferably 100% or higher, even more preferably, at least 150% or higher. These peaks can be assessed alone or in combination, or within differing percentages, as described in the previous paragraph. It is understood that the present invention also contemplates determining whether an individual does not have or will not develop cancer by ruling the individual out using the methods described herein.

In block 212, a test is performed to determine whether more samples are available for testing. If so, then control transfers to block 204 and the process repeats for the next sample. If not, then control transfers to block 214, in which output results are provided. Providing output results may comprise generating one or more reports, graphs, charts, or other record of results. Providing output results also may comprise storing results in memory, database, or other computer storage.

The process of FIG. 2A may be used to improve and modify the prediction model by comparing it to a test data set in which the pathology of individuals is known. As seen in FIG. 1B, prediction model 138 is compared to the test data set 132, and the prediction model is modified, resulting in creation of final prediction model 140. The process of FIG. 2A may then be used to perform diagnosis or prediction of cancerous activity in a population for which pathology is unknown. Alternatively, the process of FIG. 2A may be used to perform diagnosis or prediction of cancerous activity in a population for which pathology is unknown without refining the prediction model based on the test data set.

Referring now to FIG. 2B, a serum sample 152 is obtained from each individual in a population 150 for which individual pathology is unknown. The serum sample 152 is applied to mass spectrometer 130, in the manner described above, to result in generating spectral weight values for each serum sample 154. The final prediction model 140 is applied to the spectral weight values for each serum sample 154 using pattern matching as described with respect to blocks 204-210 and 214 of FIG. 2A, to result in generating a diagnosis or prediction of whether an individual has or will develop cancer, as indicated by block 156.

The specificity and sensitivity of LDA can be altered by using, for example, a simple stochastic model. It can be assumed that predictors (X) follow a multivariate normal distribution conditional on the binary outcome (Y). To predict Y for a particular value of X, the value of Y that maximizes the posterior probability of observing X, given that value of Y, can be determined. Prior probabilities for each value of Y can be assigned and can be used to control sensitivity and specificity.

For example, if a prior probability of 0 is assumed, there would be no false or true positives. If a prior probability of 1 is assumed, both false and true positive rates will be 100%. The training data can be used to estimate the parameters, mean and covariance matrix associated with each conditional distribution. Using LDA, a tuning parameter can be set that directly affects the balance between sensitivity and specificity. Cross-validation results for a range of the tuning parameter can then be used to construct receiver operating characteristic (ROC) curves.

2.4 Empirical Results

A population of 191 patients with lung or head and neck cancer and 143 control subjects was selected. The control population included a higher frequency of individuals who smoked or drank than the frequency found among the general population. Diluted serum samples were subjected to MALDI mass spectroscopy operated in a linear mode, with data acquired from 0 to 180 kd. Vansteenkiste, J. F., Eur Respir J Suppl, 34: S115-121 (2001). Information was extracted from the points along the entire mass spectra by treating the data as one continuous curve from 0 to 180 kd along the x-axis. A preferred number of spectral features to use in the LDA was selected based on peak height and those peaks which appeared to best differentiate between patient and control subjects. See Fisher, R A, Ann Eugen, 7: 179-88 (1936). For each value of P (number of features), the area under the ROC curves obtained using the cross-validation described above was calculated. This provided a function of area under the curve on the y-axis and the number of covariates on the x-axis. The area under the ROC curve is a typical one-number summary of an ROC curve.

With LDA, a tuning parameter can be set that directly affects the balance between sensitivity and specificity. See Venables, W N, “Modem Applied Statistics,” (4th Ed., NY), Springer (2002). Thus, the cross-validation results were used for a range of tuning parameters to construct receiver operating characteristic (ROC) curves. A “P” value was estimated based on the 200 simulations.

Mean false and true positive rates were obtained by considering the number of times that correct and incorrect calls were made over the 200 simulations. These rates were compared across different groups based on sex, age, disease stage, smoking history and alcohol history using the general linear methods function in “R.” See Ihaka and Gentleman, Graph Stat, 5: 299-314 (1996).

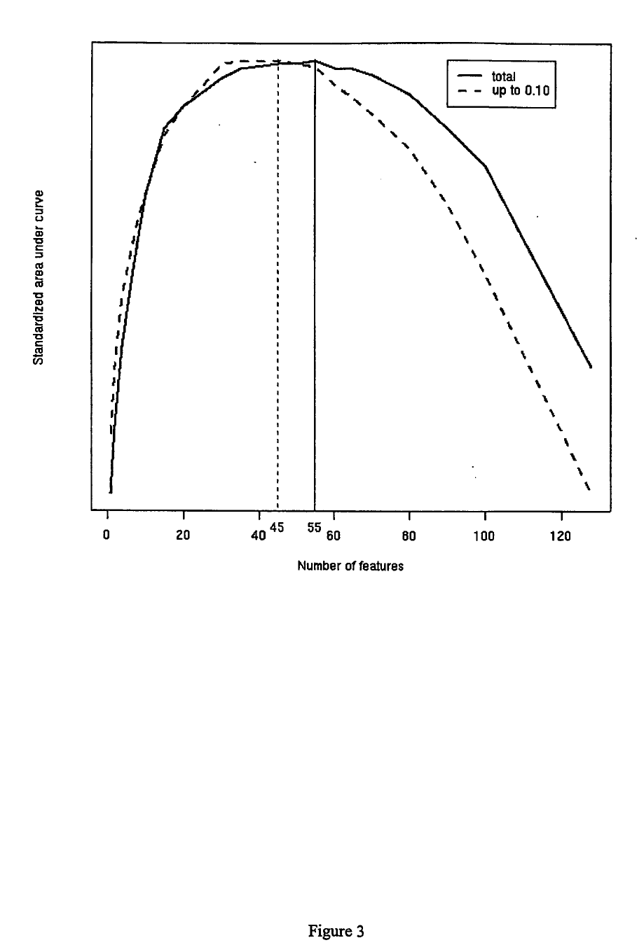

For high specificity, the area under the curve was considered for false positive rates up to 10%. These areas were plotted against the number of features used by the LDA. The maximum area under the ROC curve value occurred when 45 features were used. See FIG. 3. Thus, a feature selection procedure was defined that selects as predictors in the LDA the top 45 spectral weights in a ranking according to the absolute value of the t test.

Next, two-thirds of the data was chosen to train the procedure, and the other one third was chosen to test the procedure. By considering false- and true-positive rates in only the test set, average rates in the test set provided a measure of prediction.

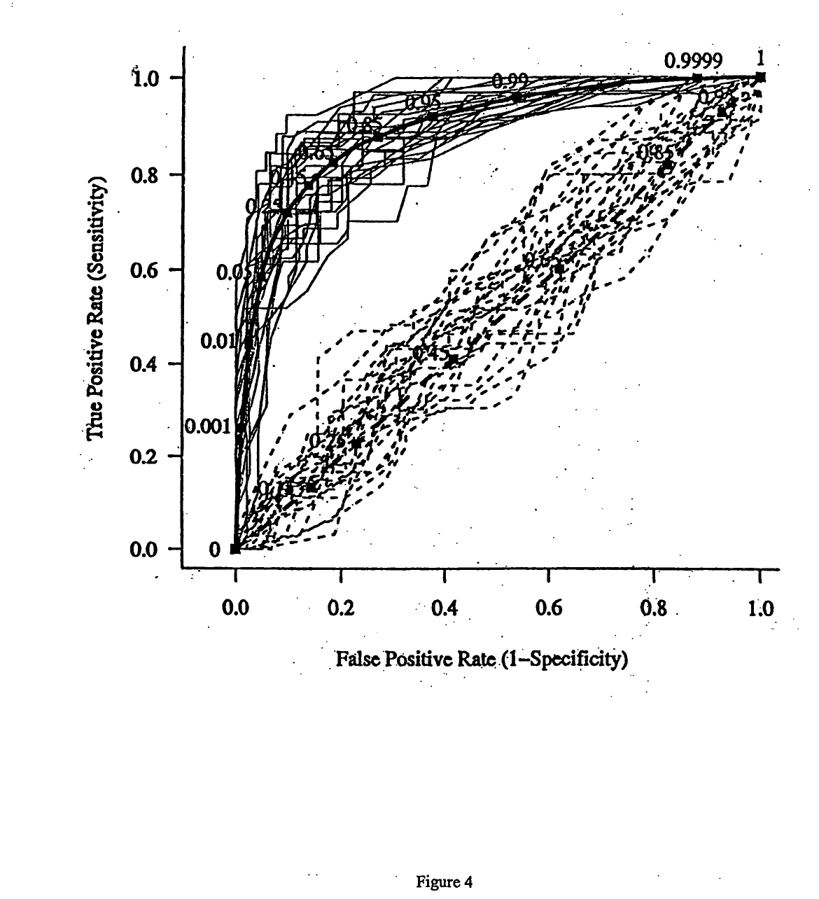

Outcomes for the test sets were predicted for the test sets on the basis of randomly chosen divisions of the data, as described above. To be sure that the predicted outcomes were not the result of mathematical artifacts, the procedure was repeated 200 times after randomly permuting the outcomes of Y. The specificity and sensitivity of each model was calculated across a range of cutoffs. An ROC curve was generated for each of the 200 permutations, and the ROC curves were averaged. See FIG. 4. The average ROC curve was computed by averaging the true-positive rate associated with each false-positive rate.

At the mean outcome with a sensitivity of 70% at a specificity of 90%, the 200 permutations never intersected with the null hypothesis (P=0.01, 95% confidence interval=0.00 to 0.02). Because these ROC curves were always calculated on data independent from the data that generated the models, they reflect what would be expected in practice, and demonstrate that this prediction model is statistically significantly better than the null hypothesis.

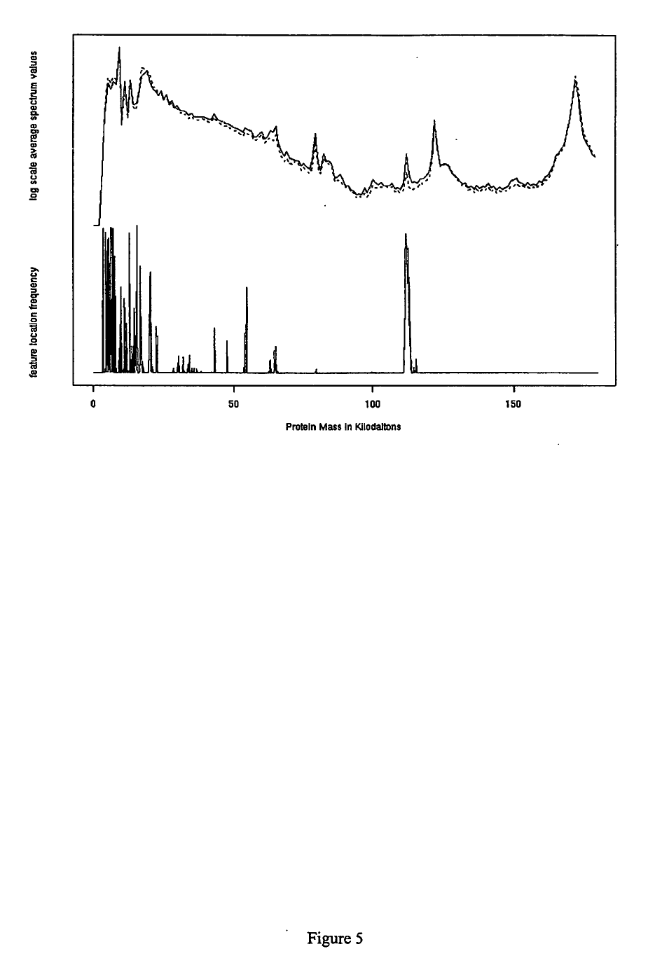

FIG. 5 is a summary of the average spectra for head and neck cancer patients and control subjects. In general, sera from the cancer patients contained more total protein than sera from control subjects. The lower portion of the figure is a histogram distribution of individual points, demonstrating the number of times the points emerged as features during 200 random divisions of the data. The most frequently appearing points correspond to positions where peaks appeared to disappear in the head and neck cancer samples. One particular peak, at approximately 111 kd, was different between sera from case patients and control subjects in all 200 simulations. Other peaks generally useful in the analysis of the present invention are at approximately 5, 10, 12, 15, 20, 45, 47, 54 and 64 kd. Such peaks represent molecules that are serum markers for cancer, particularly upper aerodigestive tract cancer such as head and neck or lung cancer, as described herein. See Srinivas et al., Clin. Chem. 48, 1160-69 (2002); Petricoin et al., Nat. Rev. Drug Discov. 1, 683-95 (2002); Pardanani et al., Mayo Clin. Proc. 7, 1185-96 (2002).

The present invention provides diagnosing a subject with head, neck or lung cancer by generating mass spectral data from the serum or blood of the subject and matching this data with the data generated from one or more subjects with head, neck or lung cancer. A “match” is made with one or more peaks. Peaks are matched as described above. Preferably two or more peaks are matched, more preferably, three, four, five, six, seven, eight, nine, or ten or more peaks are matched. The invention also provides diagnosing head, neck or lung cancer in a subject by identifying one or more proteins in the blood or serum of the subject. The proteins are generally within 2% of the identifying spectral weights (i.e., 111, 5, 10, 12, 15, 20, 45, 47, 54 or 64 kd), more preferably, within 1.5%, even more preferably, within 1% and, yet more preferably, within 0.5%. Preferably two or more proteins are identified, more preferably, three, five, seven or ten or more proteins are identified within the parameters described. The above methods of diagnosing a subject also apply for monitoring a subject previously diagnosed for recurrence. The model described herein, which was developed for head and neck cases and healthy controls, and using an optimal cutoff that had 73% sensitivity and 90% specificity, was applied to lung cancer patients. For the same example investigation, Table 1 presents the percentage sensitivity for each diagnosis and the number of actual cases.

TABLE 1

| Diagnosis | Percent | Number |
|---|---|---|
| acute pneumonia;* negative for tumor | 0 | 7 |
| adenocarcinoma | 34 | 50 |
| large cell carcinoma | 40 | 5 |
| other carcinoma** | 25 | 4 |
| squamous cell carcinoma | 52 | 33 |

*and other inflammatory conditions
**two cases of small cell, one lymphoma, and one carcinoid

Given the fundamental histologic diversity of the diagnoses in Table 1 and the fact that the model was developed from head and neck cases, the sensitivity of prediction was successful. Specifically, the sensitivity for lung SCC was 52%, lung adenocarcinoma 34%, and large-cell carcinoma 40% when the false positive rate was 10%. Moreover, when the model of the subject invention was applied to 7 individuals who had acute pneumonia or other inflammatory lung conditions but did not have cancer, all were scored as negative.

Thus, the present invention shows that certain comorbid conditions do not raise the false positive rate. In addition, no differences in prediction were found based on disease stage, race, ethnicity, sex or smoking history in either head and neck or lung cancer populations.

2.5 Representing Prediction as a Regression Problem

For purposes of further understanding the approach herein, the prediction problem presented herein can be represented as a regression problem. In the regression view, the problem is to estimate the expected value of Y, given observation of the covariates Xj. In statistical notation, the regression problem is expressed as:
μ(Y|X₁, . . . X_{?})=E[Y|X₁, . . . , X_{?}]
Therefore, the goal of the approach herein is to estimate
μ(Y|X₁, . . . X_{?})
using the observed data, is denoted as with yᵢ and xᵢⱼ for i=1, . . . ,N andj=1, . . . ,?.

In solving the foregoing, the usual approach of logistic regression is not appropriate, since there are many more covariates than outcomes. The resulting fit would produce perfect predictability, but only as a mathematical artifact. Furthermore, there is no science justifying the logistic scale linear relationship assumption. Finally, because in this problem correct predictions are more important than the interpretation of model parameters, the typical linear regression model has no advantages. Any procedure that can reliably predict the outcomes is considered useful, regardless of interpretability of parameters. Thus, the computational process described herein is best viewed as a classification, in which a process that can reliably predict Y given the spectra X is sought.

3.0 Implementation Mechanisms—Hardware Overview

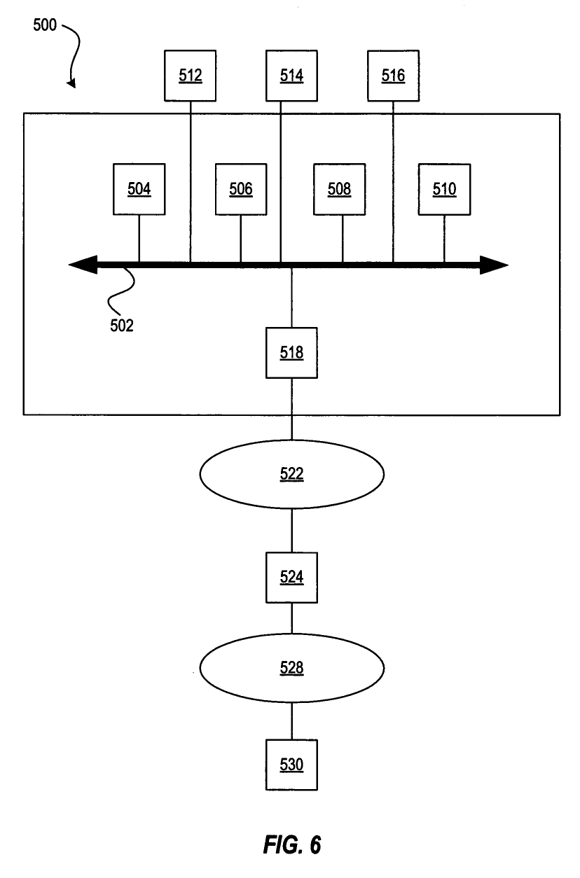

FIG. 6 is a block diagram that illustrates a computer system 500 upon which an embodiment of the invention may be implemented. Computer system 500 includes a bus 502 or other communication mechanism for communicating information, and a processor 504 coupled with bus 502 for processing information. Computer system 500 also includes a main memory 506, such as a random access memory (“RAM”) or other dynamic storage device, coupled to bus 502 for storing information and instructions to be executed by processor 504. Main memory 506 also may be used for storing temporary variables or other intermediate information during execution of instructions to be executed by processor 504. Computer system 500 further includes a read only memory (“ROM”) 508 or other static storage device coupled to bus 502 for storing static information and instructions for processor 504. A storage device 510, such as a magnetic disk, optical disk, solid-state memory, or the like, is provided and coupled to bus 502 for storing information and instructions.

Computer system 500 may be coupled via bus 502 to a display 512, such as a cathode ray tube (“CRT”), liquid crystal display (“LCD”), plasma display, television, or the like, for displaying information to a computer user. An input device 514, including alphanumeric and other keys, is coupled to bus 502 for communicating information and command selections to processor 504. Another type of user input device is cursor control 516, such as a mouse, trackball, stylus, or cursor direction keys for communicating direction information and command selections to processor 504 and for controlling cursor movement on display 512. This input device typically has two degrees of freedom in two axes, a first axis (e.g., x) and a second axis (e.g., y), that allows the device to specify positions in a plane.

The invention is related to the use of computer system 500 for predicting head, neck and lung cancers. According to one embodiment of the invention, predicting head, neck and lung cancers is provided by computer system 500 in response to processor 504 executing one or more sequences of one or more instructions contained in main memory 506. Such instructions may be read into main memory 506 from another computer-readable medium, such as storage device 510. Execution of the sequences of instructions contained in main memory 506 causes processor 504 to perform the process steps described herein. In alternative embodiments, hard-wired circuitry may be used in place of or in combination with software instructions to implement the invention. Thus, embodiments of the invention are not limited to any specific combination of hardware circuitry and software.

The term “computer-readable medium” as used herein refers to any medium that participates in providing instructions to processor 504 for execution. Such a medium may take many forms, including but not limited to, non-volatile media, volatile media, and transmission media. Non-volatile media includes, for example, optical or magnetic disks, solid state memories, and the like, such as storage device 510. Volatile media includes dynamic memory, such as main memory 506. Transmission media includes coaxial cables, copper wire and fiber optics, including the wires that comprise bus 502. Transmission media can also take the form of acoustic or light waves, such as those generated during radio wave and infrared data communications.

Common forms of computer-readable media include, for example, a floppy disk, a flexible disk, hard disk, magnetic tape, or any other magnetic medium, a CD-ROM, any other optical medium, solid-state memory, punch cards, paper tape, any other physical medium with patterns of holes, a RAM, a PROM, and EPROM, a FLASH-EPROM, any other memory chip or cartridge, a carrier wave as described hereinafter, or any other medium from which a computer can read. Various forms of computer readable media may be involved in carrying one or more sequences of one or more instructions to processor 504 for execution.

Computer system 500 may also include a communication interface 518 coupled to bus 502. Communication interface 518 provides a two-way data communication coupling to a network link 520 that is connected to a local network 522. For example, communication interface 518 may be an integrated services digital network (“ISDN”) card or a modem to provide a data communication connection to a corresponding type of telephone line. As another example, communication interface 518 may be a network card (e.g., and Ethernet card) to provide a data communication connection to a compatible local area network (“LAN”) or wide area network (“WAN”), such as the Internet. Wireless links may also be implemented. In any such implementation, communication interface 518 sends and receives electrical, electromagnetic or optical signals that carry digital data streams representing various types of information.

Network link 520 typically provides data communication through one or more networks to other data devices. For example, network link 520 may provide a connection through local network 522 to a host computer 524 or to data equipment operated by an Internet Service Provider (“ISP”). ISP in turn provides data communication services through the worldwide packet data communication network now commonly referred to as the “Internet” 528. Local network 522 and Internet 528 both use electrical, electromagnetic or optical signals that carry digital data streams. The signals through the various networks and the signals on network link 520 and through communication interface 518, which carry the digital data to and from computer system 500, are exemplary forms of carrier waves transporting the information.

Computer system 500 can send messages and receive data, including program code, through the network(s), network link 520 and communication interface 518. In the Internet example, a server 530 might transmit a requested code for an application program through Internet 528, host computer 524, local network 522 and communication interface 518. In accordance with the invention, one such downloaded application provides for predicting head, neck and lung cancers as described herein.

The received code may be executed by processor 504 as it is received, and/or stored in storage device 510, or other tangible computer-readable medium (e.g., non-volatile storage) for later execution. In this manner, computer system 500 may obtain application code and/or data in the form of an intangible computer-readable medium such as a carrier wave, modulated data signal, or other propagated carrier signal.

4.0 Extensions and Alternatives

In the foregoing specification, the invention has been described with reference to specific embodiments and examples thereof It will, however, be evident that various modifications and changes may be made thereto without departing from the broader spirit and scope of the invention. The specification and drawings are, accordingly, to be regarded in an illustrative rather than a restrictive sense.

All references cited herein are herein incorporated by reference in their entireties.

1. A computer-readable medium having stored thereon a data structure for storing a cancer screening model, wherein the cancer screening model comprises a pattern of cancer predictor spectral weight values corresponding to a plurality of identifying spectral weights selected from the group consisting of 5, 10, 12, 15, 20, 45, 47, 54, 64, and 111 kd, and wherein the data structure comprises a plurality of data fields, each data field storing a spectral weight value corresponding to an identifying spectral weight.

2. The computer-readable medium of claim 1 wherein at least one of the stored spectral weight values corresponds to the identifying spectral weight of 111 kd.

3-4. (canceled)

5. The computer-readable medium of claim 1 wherein the plurality of data fields comprises:
a first data field storing a first spectral weight value corresponding to 5 kd;
a second data field storing a second spectral weight value corresponding to 10 kd;
a third data field storing a third spectral weight value corresponding to 12 kd;
a fourth data field storing a fourth spectral weight value corresponding to 15 kd;
a fifth data field storing a fifth spectral weight value corresponding to 20 kd;
a sixth data field storing a sixth spectral weight value corresponding to 45 kd;
a seventh data field storing a seventh spectral weight value corresponding to 47 kd;
an eighth data field storing an eighth spectral weight value corresponding to 54 kd;
a ninth data field storing a ninth spectral weight value corresponding to 64 kd; and
a tenth data field storing a tenth spectral weight value corresponding to 111 kd.

6. A method of generating a cancer screening model for predicting upper aerodigestive tract cancer, comprising steps of:
(a) comparing a first set of spectral weight values obtained from biological samples from a first population of individuals to a second set of spectral weight values obtained from biological samples from a second population of individuals,
wherein individuals in the first population are at high risk for developing an upper aerodigestive tract cancer but are clinically determined not to have an upper aerodigestive tract cancer; and
wherein individuals in the second population are clinically determined to have an upper aerodigestive tract cancer; and
(b) based on step (a), generating a cancer screening model which comprises a pattern of a plurality of cancer predictor spectral weight values which differentiate individuals of the first population from individuals of the second population and which correspond to identifying spectral weights selected from the group consisting of 5, 10, 12, 15, 20, 45, 47, 54, 64, and 111 kd.

7. The method of claim 6 wherein individuals in the second population are clinically determined to have a lung cancer.

8-12. (canceled)

13. The method of claim 6 wherein individuals in the second population are clinically determined to have a head and neck cancer.

14. (canceled)

15. The method of claim 6 wherein the biological samples comprise serum.

16. The method of claim 6 wherein the biological samples comprise bronchial lavage samples.

17. The method of claim 6 wherein the biological samples comprise sputum.

18. The method of claim 6 wherein the biological samples comprise biopsy samples.

19. The method of claim 6 further comprising generating the first set of spectral weight values.

20. The method of claim 6 further comprising generating the second set of spectral weight values.

21. The method of claim 6 further comprising generating the first and second sets of spectral weight values.

22. The method of claim 6 wherein determination of the presence or absence of an upper aerodigestive tract cancer is based on a clinical history and a physical examination.

23. (canceled)

24. A computer-readable medium product storing data for use in predicting upper aerodigestive tract cancer in an individual, said computer-readable medium product made by a method comprising steps of:
(a) comparing a first set of spectral weight values obtained from biological samples from a first population of individuals to a second set of spectral weight values obtained from biological samples from a second population of individuals,
wherein individuals in the first population are at high risk for developing an upper aerodigestive tract cancer but are clinically determined not to have an upper aerodigestive tract cancer; and
wherein individuals in the second population are clinically determined to have an upper aerodigestive tract cancer; and
(b) based on step (a), generating a cancer screening model which comprises a pattern of a plurality of cancer predictor spectral weight values which differentiate individuals of the first population from individuals of the second population and which correspond to identifying spectral weights selected from the group consisting of 5, 10, 12, 15, 20, 45, 47, 54, 64, and 111 kd.; and
(c) storing information corresponding to the cancer screening model on a computer-readable medium.

25. A method of predicting an upper aerodigestive tract cancer in an individual, comprising steps of:
(a) comparing test spectral weight values obtained from a biological sample from the individual to cancer predictor spectral weight values in a cancer screening model comprising a plurality of cancer predictor spectral weight values corresponding to identifying spectral weights selected from the group consisting of 5, 10, 12, 15, 20, 45, 47, 54, 64, and 111 kd; and
(b) identifying the individual as having or as likely to develop an upper aerodigestive tract cancer if a plurality of the test spectral weight values are within 25% or higher of their corresponding cancer predictor spectral weight values.

26. The method of claim 25 wherein at least one of the plurality or cancer predictor spectral weight values corresponds to the identifying spectral weight value of 111 kd.

27-42. (canceled)

43. A computer-readable medium storing computer-executable instructions for performing a method comprising steps of:
(a) comparing test spectral weight values obtained from a biological sample from the individual to cancer predictor spectral weight values in a cancer screening model comprising a plurality of cancer predictor spectral weight values corresponding to identifying spectral weights selected from the group consisting of 5, 10, 12, 15, 20, 45, 47, 54, 64, and 111 kd; and
(b) identifying the individual as having or as likely to develop an upper aerodigestive tract cancer if a plurality of the test spectral weight values are within 25% or higher of their corresponding cancer predictor spectral weight values.

44. (canceled)
